(19) United States
(12) Patent Application Publication
(10) Pub. No.: US 2012/0289913 A1
(43) Pub. Date: Nov. 15, 2012

(54) Device for providing a vacuum for medical vacuum wound treatment

(76) Inventors: Axel Eckstein, Heidenheim (DE); Juergen Hofstetter, Heidenheim (DE)

(21) Appl. No.: 13/467,088

(22) Filed: May 9, 2012

Related U.S. Application Data
(60) Provisional application No. 61/457,841, filed on Jun. 17, 2011.

(30) Foreign Application Priority Data
May 13, 2011 (DE) 10 2011 075 842.9

(51) Int. Cl. A61M 1/00; A61M 3/02

(52) U.S. Cl. 604/305; 604/319

(57) ABSTRACT

A portable device (2) for providing a vacuum for medical vacuum treatment of wounds has a suction pump (90), which generates a vacuum and is located in a housing part (4) of the device as well as a container (10) for receiving body fluids. A programmable electronic control device (100) can drive the suction pump (90), taking into consideration predetermined parameters. A sensor device (120) is provided by means of which the electronic control device (100) can detect when the container (10) is removed from the housing part (4) during vacuum regulation operation, wherein the sensor device (120) emits a signal, on the basis of which the electronic control device (100) detects removal of the container (10) and the electronic control device (100) is designed in such a fashion that it deactivates the suction pump (90) in this case.

True translation of 61/457,841 as filed on Jun. 17, 2011 and of Ser. No. 13/467,088 as filed on May 9, 2012.

The invention concerns a device that can be carried on the body of a user for providing a vacuum for medical vacuum treatment of wounds on the body of a person or of an animal, comprising a suction pump, which generates a vacuum and is located in a first housing part of the device, a container for receiving body fluids, in particular wound exudates extracted from the body by suction, wherein the container can be detachably mounted to the first housing part of the device and can be evacuated by the suction pump in the mounted state, and wherein a connection is provided for a suction line that leads to the body such that vacuum communication can be established between the suction pump, the container, and the suction line that leads to the body, a programmable electronic control device, which controls the suction pump thereby at least taking into consideration predetermined and/or predeterminable parameters and pressure values measured by the pressure sensor. The above-mentioned definition “portable device” means that the patient can carry the device along so that he/she is mobile and his/her wound can nevertheless be permanently treated, i.e. without interruption. The portable device may thereby be held on the body of the patient and be carried along by means of any fastening means, in particular and preferably in the form of a flexible belt or shoulder strap. A portable device of the above-mentioned type may naturally also be used for stationary operation, i.e. detached from the body of the patient. In this case, it may e.g. be mounted to a hospital bed or be deposited next to the hospital bed.

Vacuum wound treatment devices have been described many times, in particular, in US 2004/0073151 A1, WO 2009/047524 A2, WO 2007/030599 A2 or EP 1 905 465 A1, EP 777 504 B and in DE 10 2009 038 130 A1 and DE 10 2009 038 131 A1 of the assignee.

In devices of this type for vacuum treatment of wounds, a suction pump communicates with the wound or the wound area via a suction line, wherein a wound dressing with an air-tight cover material is provided for air-tight sealing of the wound and the wound area, such that a vacuum can be generated in the wound region and fluids can be extracted by suction from the wound region into the above-mentioned container.

The term vacuum in connection with the present invention defines an air pressure that is lower than the ambient air pressure (atmospheric air pressure), in particular, inside a wound dressing. The cover material of a wound dressing for air-tight sealing of a wound region must therefore be designed in such a fashion that it withstands the pressure difference that is established such that a vacuum can actually be applied to and maintained in the wound region. The wound dressing and the cover material are, however, typically flexible to a certain degree. In the field of vacuum therapy for the treatment of wounds, the vacuum is quantitatively defined as the pressure difference between ambient air pressure and the air pressure applied below the cover material. In the field of vacuum therapy, this pressure difference is typically at most 250 mmHg (mm mercury column) (1 mm Hg=1 Torr.=133.322 Pa). This vacuum range of up to maximally 250 mmHg has turned out to be suitable for wound healing. A preferred vacuum range is between 10 and 150 mmHg.

For typical vacuum treatment, the vacuum that is applied to the wound using the device can either be kept substantially constant with time or can be varied with time, in particular, in cycles, which can be realized by a correspondingly designed and programmed control device for the vacuum-generating device, in particular, in dependence on further parameters.

An advantageously flexible suction line, e.g. in the form of a drainage hose, is provided for applying a vacuum and advantageously also for extracting body fluids, the drainage hose communicating at one end with the wound area or the wound region via a so-called port in the area of the wound cover material, and at the other end communicating with the above-mentioned container for receiving body fluids, or with the vacuum generating device.

In addition to vacuum wound treatment, the present device may also be used for other applications for providing a vacuum for medical applications, in particular, extraction of any body fluids by suction, in the field of medical incontinence management, in the field of care of stoma patients or in the field of extraction of wound exudates, if necessary, thereby using rinsing liquids and also without application of a vacuum over considerable time periods.

Based on a portable device of this type for providing a vacuum for medical applications, it is the underlying purpose of the present invention to further optimize the user friendliness and operational safety such that a technically less skilled user or patient is given the feeling that she/he can safely control operation of the device.

In accordance with the invention, this object is achieved with a device of the above-mentioned type in that a sensor device is provided for determining, by means of the electronic control device, when the container is removed from the first housing part during vacuum regulation operation, wherein the sensor device emits a signal by means of which the electronic control device detects that the container has been removed and that the electronic control device is designed to activate the suction pump in this case.

Deactivation of the suction pump in this connection means interruption of the operation of the device until the device is restarted, which can be triggered in a simple fashion, i.e. the instantaneously running pressure regulation operation and therefore driving of the pump are permanently interrupted until the user initiates restart and therefore a new operating cycle, or until the container, in particular, a new, unused container has been arranged in its intended mounting position. With the present invention, it has turned out that the main cause of maloperation of devices for vacuum wound treatment that can be carried on the body of a patient consists in that the container for receiving body fluids is detached from the first housing part of the device containing the suction pump and the electronic control components such that in consequence thereof, control-technologically undifferentiated states are generated. When e.g. starting from an instantaneously active vacuum control or regulation operation, the container is detached from the first housing part of the portable device, the control could consequently cause the electronic control device to drive the suction pump towards increasing suction power, which is often accompanied by an increased noise, which can render the user insecure and moreover may require the user to take further control measures. This situation can also arise when a container that has been completely or almost completely filled with liquid is deliberately replaced by a new container.

The inventive design of the device, however, ensures that, on the basis of a running vacuum regulation operation, the suction pump that generates a vacuum is deactivated by the electronic control device as soon as the container is removed, which is detected by the sensor device in cooperation with the electronic control device. In this fashion, no undefined states are generated which, in combination with possibly introduced control measures, could confuse the user. The user can instead get used to restart operation of the device as usual through familiar actuation after exchange of the container, wherein automatic restart may alternatively also be performed as soon as the released container or a new container has been appropriately mounted to the first housing part. In this case, the same sensor device advantageously operates quasi in the opposite direction in that it signals to the control device that the container has been rearranged such that the control device can initiate restart of the previously run program.

The present invention is particularly important for mobile use of portable devices of the present type, since patients being provided with such a portable device have to rely on themselves, remote from a clinical facility and for this reason, the variety of states of complex devices should also be reduced to a minimum, which the patient must comprehend and also react to i.a. by taking operating measures. This is considerably facilitated for the technically unskilled user or patient in that she/he is always presented with the same state, namely operation deactivated, when the container is detached or removed, to which state she/he always has to react in the same fashion, e.g. by renewed start of operation or remounting of the same or a fresh container. The inventive design therefore facilitates the overall handling of the device, thereby also reducing the susceptibility to misuse and thereby increasing the overall operational safety.

The above-mentioned sensor device may basically be realized in many ways. The above reference to the signal merely means cooperation between the sensor device and the electronic control device. In the simplest case, the sensor device may therefore comprise a switch which is actuated when the container is arranged in its intended mounting position on the first housing part and is opened again when the container is removed or vice versa. The signal emitted to the sensor device then consists in a change of state which is received by a sensor input, is processed in the electronic control device, is correspondingly recognized and triggers the further control processes, i.e. at least deactivation of the suction pump.

In accordance with an embodiment of the invention, the sensor device has an actuator that is pretensioned towards the container, is mechanically actuated by the container, and is displaced against the pretensioning force when the container is arranged. This actuator may then act on further actuating devices, it may, in particular, mechanically actuate a switch, which is recognized as such by the electronic control device.

In accordance with a further embodiment, the sensor device may comprise an electric contact sensor, in which a tactile contact is triggered when the container is arranged in its intended mounting position on the first housing part.

In accordance with a further embodiment, the sensor device may be designed to be magnetically or electromagnetically active. It comprises a magnetically or electromagnetically actuated component, in particular, with a relay function, the switching state of which is influenced by the arrangement or non-arrangement of the container in its intended mounting position.

In accordance with a further embodiment, the sensor device may also be designed to act in an optoelectronic fashion such that the presence or absence of the container in its intended mounting position is detected by optoelectronic means, i.e. in the broadest sense is optically detected.

In accordance with a further embodiment, the sensor device may be designed to be capacitively active in that the change of a capacitively influenced variable is measured, which changes when the container is removed from the first housing part during vacuum regulation operation.

In accordance with a further particularly important embodiment of the invention, the sensor device is formed by a pressure sensor which is arranged in a line section between the container and the suction pump for measuring the pressure and the electronic control device is designed in such a fashion that it deactivates the suction pump when, starting from running vacuum regulation operation, a pressure change rate (Δp/Δt) determined by the signals of the pressure sensor exceeds a predetermined threshold value in the direction of decreasing vacuum.

In this case, removal of the container is detected by a pressure sensor which detects the associated abruptly decreasing vacuum, i.e. an abruptly increasing absolute pressure. The electronic control device can determine this abrupt pressure increase by means of the pressure change rate (Δp/Δt) by a threshold value comparison. This variant of the invention turns out to be advantageous in that no further device components must be provided, since the mentioned pressure sensor is anyway arranged between the container and the suction pump for continuous or quasi continuous pressure detection.

The above-mentioned threshold value for the pressure change rate (Δp/Δt) in the direction of decreasing vacuum, i.e. in the direction of an increase of the absolute pressure, is advantageously at least 40 mmHg/s (mm mercury column per second). In particular, it may be implemented in the programmable electronic control device at 45, 50 or 55 mmHg/s.

It has also turned out to be advantageous to provide an output or display device for generating a signal which visually or acoustically communicates an abrupt pressure increase with a pressure change rate above the threshold value and therefore deactivation of the suction pump. In this fashion, the user or patient may be informed that she/he should restart operation of the device as usual, in particular, after remounting of the container or after replacement by a new container.

In a further development of the invention, it may be advantageous to provide an output or display device, which is designed for wireless transmission of a signal to an external receiver, the signal communicating the abrupt pressure increase with a pressure change rate above the threshold value and thereby communicating deactivation of the suction pump to an external receiver. The external receiver may e.g. be a ward center in a hospital or a care control center or the like such that deactivation of the pressure regulation operation or of the suction pump is communicated to that center where further steps can be taken, in particular, for patients in need of care.

As mentioned above, it may be advantageous for the sensor device to quasi operate in an opposite direction and detect remounting of the container such that pressure regulation operation can be automatically restarted. In accordance with this inventive idea, the sensor device and the electronic control device are designed in such a fashion that (re)mounting of the container in its intended position on the first housing part is detected and, based on a previously performed deactivation of the suction pump, the previously stopped pressure regulation operation is automatically started again in this case.

The inventive device may furthermore comprise a ventilation valve which can be controlled by the control device and can be connected to the wound region via a venting line that is provided in addition to the suction line, such that the wound region can be vented with air from the outside.

The invention has turned out to be particularly advantageous in a device which is further characterized by a device, which can be controlled by the electronic control device, for supplying a rinsing liquid or another fluid and which can be connected to the wound region via a rinsing line that is provided in addition to the suction line such that rinsing liquid or another fluid, in particular containing therapeutically active components, can be supplied to the wound region. In this case, it has turned out to be particularly advantageous for the electronic control device to be further designed in such a fashion that it also deactivates the device for supplying a rinsing liquid or another fluid when the suction pump is deactivated. It has turned out that the operational safety can be considerably improved when, during container change or any other operational disturbance, the device for supplying a rinsing liquid is permanently deactivated together with the suction pump until the device is restarted.

There are devices for vacuum wound treatment of this type such as e.g. EP 777 504 A, in which a fill level sensor is provided to detect whether the container is full and must be replaced by a new container. When the container is detected to be full, the suction pump is deactivated, i.e. the pressure regulation operation is interrupted. Within the scope of the present invention, this turned out to be disadvantageous since exactly for portable devices, fill level monitoring of conventional containers during mobile operation often provides incorrect information. For example, due to movement of the user, e.g. bending movement, a state “container is full” is temporarily detected, which is actually not the case. Deactivation of the suction pump moreover generally causes premature reduction of the vacuum. It is often the case, that an unused container is not available or that such measures of care must be postponed. A further development of the present invention therefore proposes to design the electronic control device in such a fashion that it does not deactivate the suction pump when the container has been correctly or incorrectly detected to be full, but continues the predetermined vacuum regulation operation. In this fashion, it is ensured that the vacuum regulation operation is not interrupted too early or erroneously. Even when it has been reliably detected that the container actually needs to be changed, it has turned out to be advantageous to continue normal vacuum regulation operation via the electronic control device, i.e. the suction pump maintains the vacuum on the container as predetermined by the control technology. When the container has been removed from the first housing part, i.e. the base of the device, the suction pump is permanently deactivated.

Irrespective thereof, the electronic control device is advantageously designed in such a fashion that it does not deactivate, i.e. permanently stop, the suction pump such that it must be started again but continues the predetermined vacuum regulation operation in case of a vacuum increase, i.e. a decrease of the absolute pressure in the line section between the container and the suction pump, which suggests, in particular, an increasing fill level of the container or other, in particular, temporary undefined states or disturbances, in particular due to movement of the user. In accordance with this inventive idea, the vacuum regulation operation is only deactivated when it has been detected that the container has been removed from the first housing part of the device. Following the above inventive idea, an increasing vacuum (decreasing absolute pressure), in particular, an abruptly increasing vacuum, may suggest that the container is full, in particular, when a filter is used in the region of a container outlet towards the line section to the suction pump, since the filter is permeable to air but not to liquid. When the liquid level in the container increases up to or beyond this filter, the filter is increasingly blocked and causes the vacuum to increase in the line section between the container and the suction pump, which is again detected by the pressure sensor.

The invention also concerns a method comprising the features of claim 18.

Further features, details and advantages of the invention can be extracted from the enclosed claims and the drawing and the subsequent description of a preferred embodiment of the invention. In the drawing:

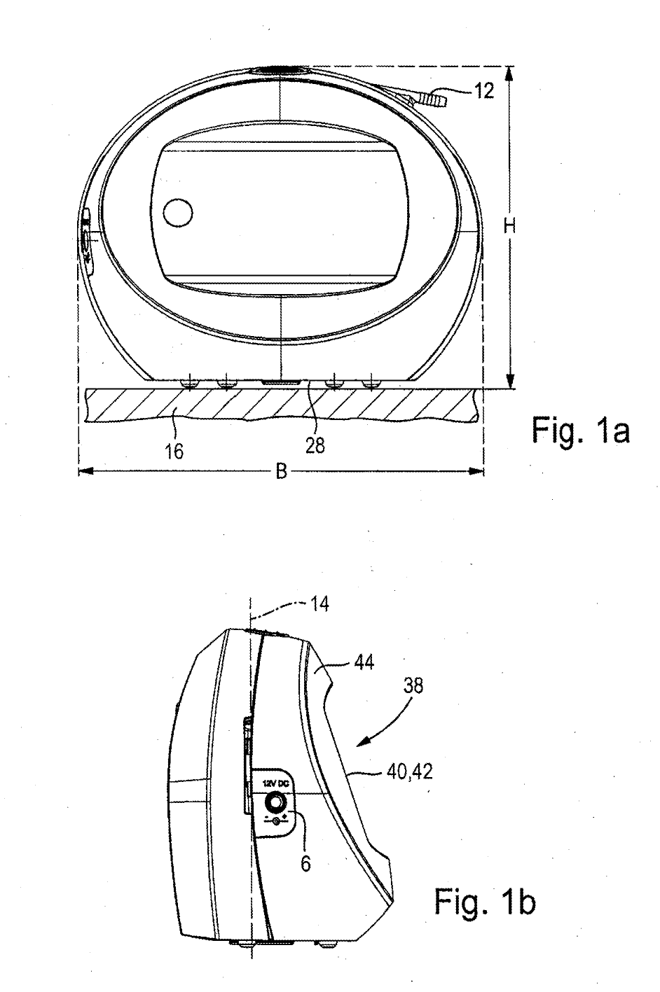

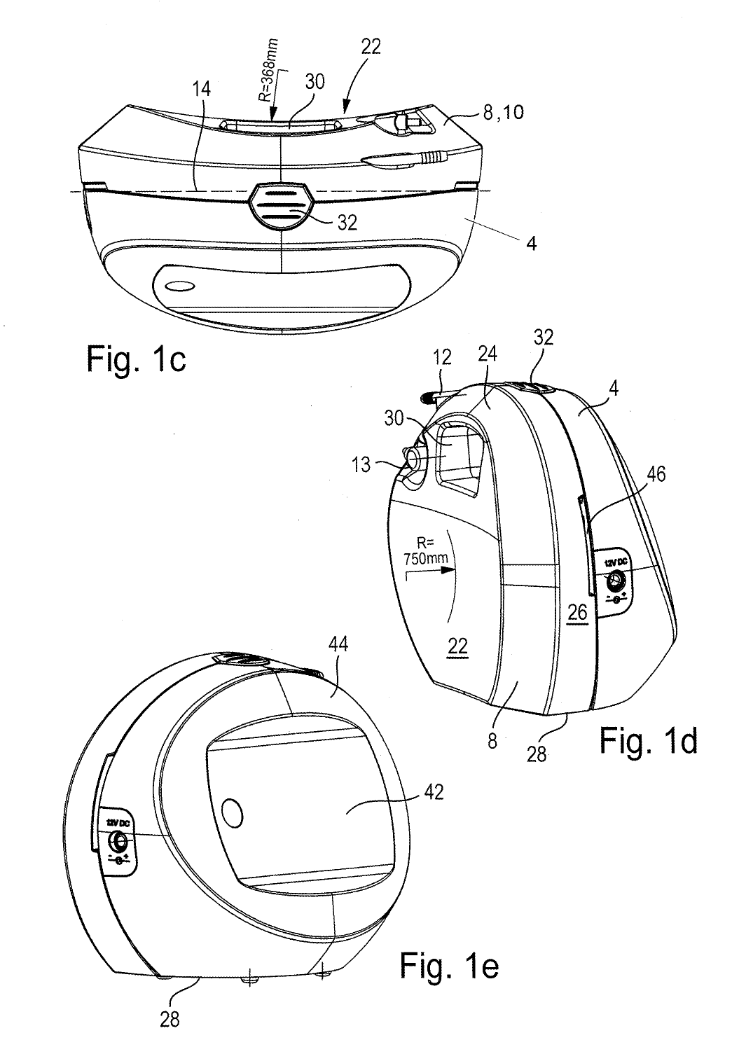

FIGS. 1a through e show different views of a preferred embodiment of a device that can be carried on the body for providing a vacuum for medical applications;

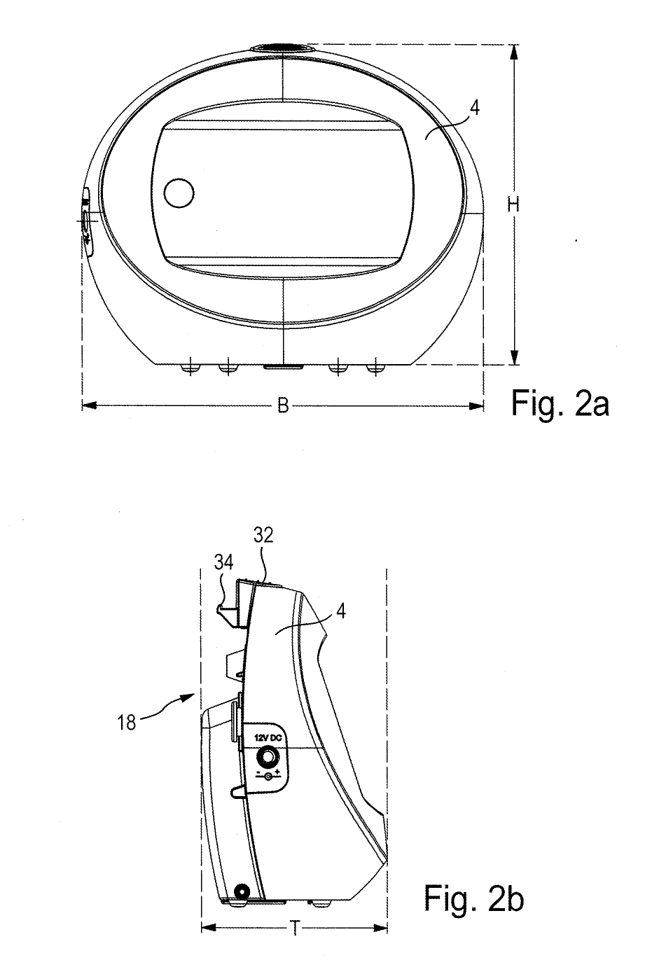

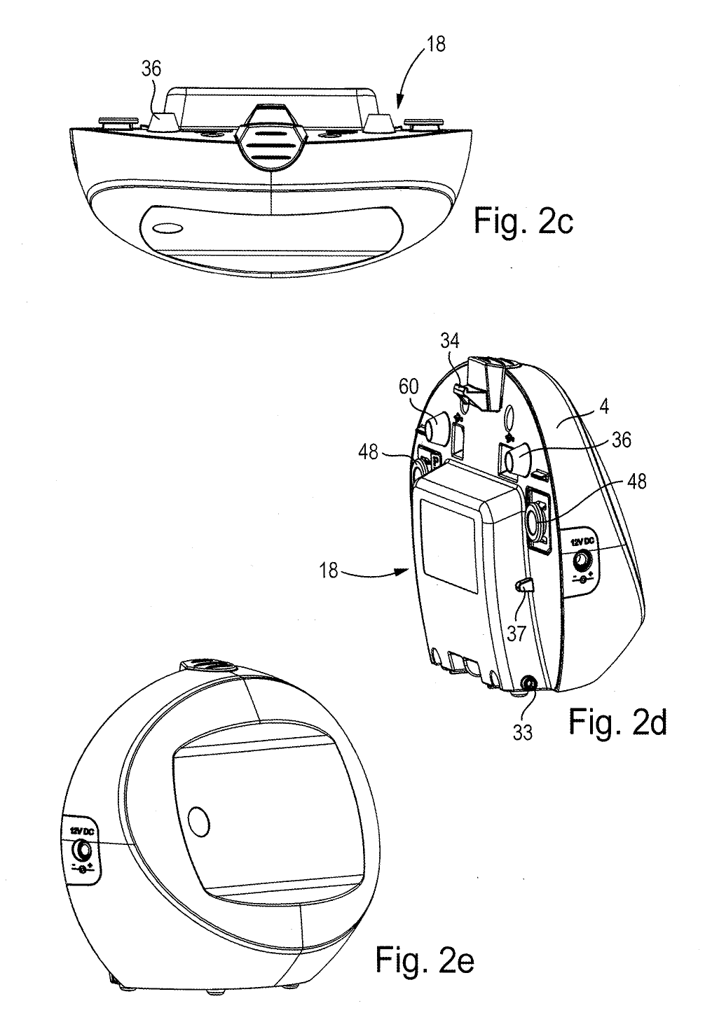

FIGS. 2a through e show different views of a first housing part of the device according to FIG. 1, comprising a vacuum-generating device and control components;

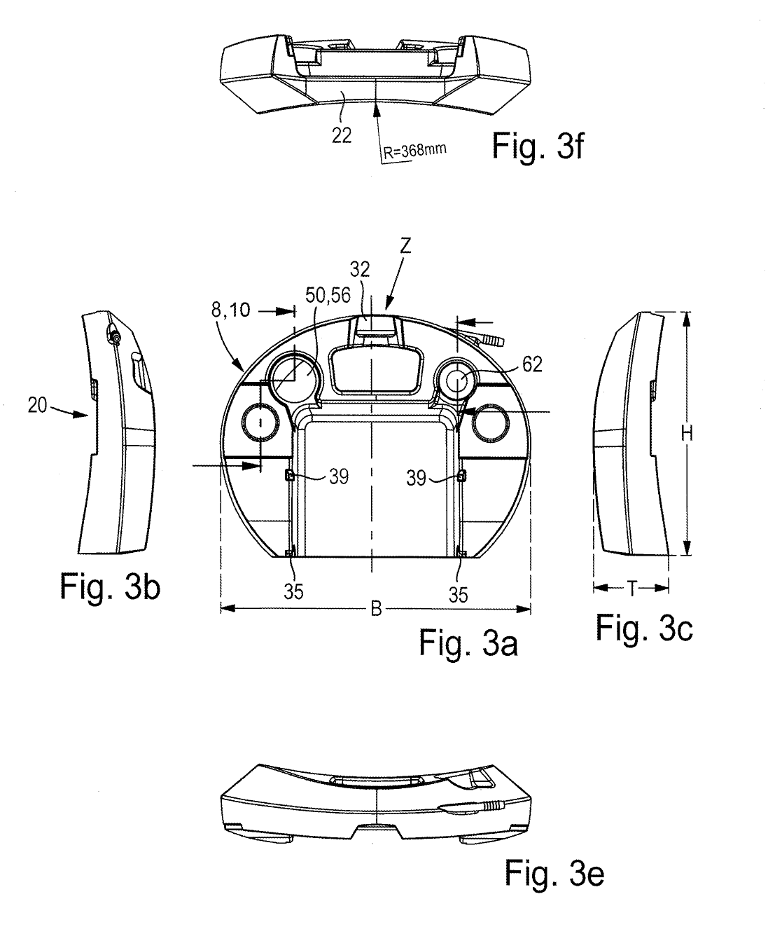

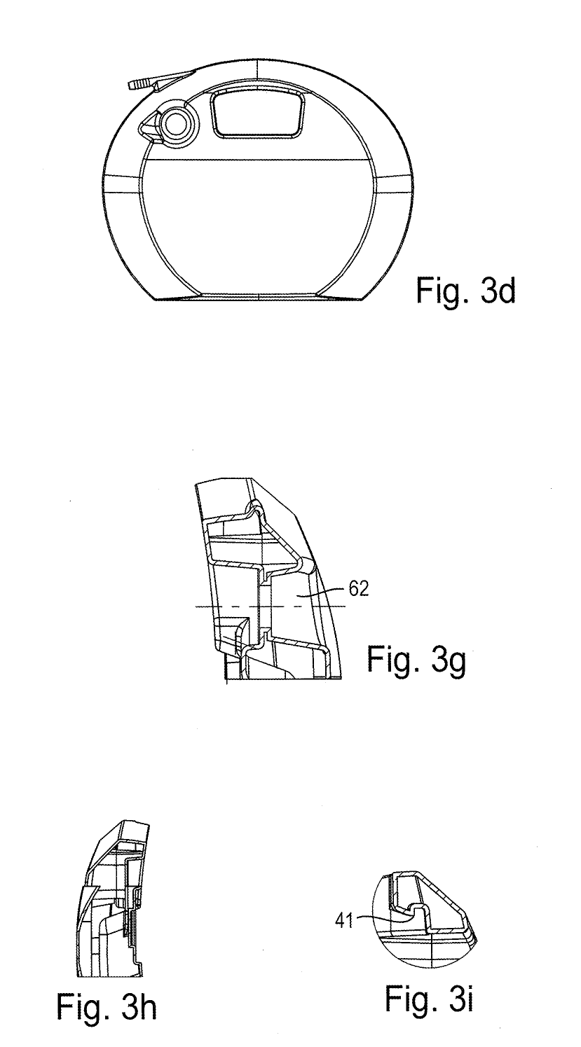

FIGS. 3a through i show different views of a second housing part of the device according to FIG. 1, which forms a container for receiving body fluids;

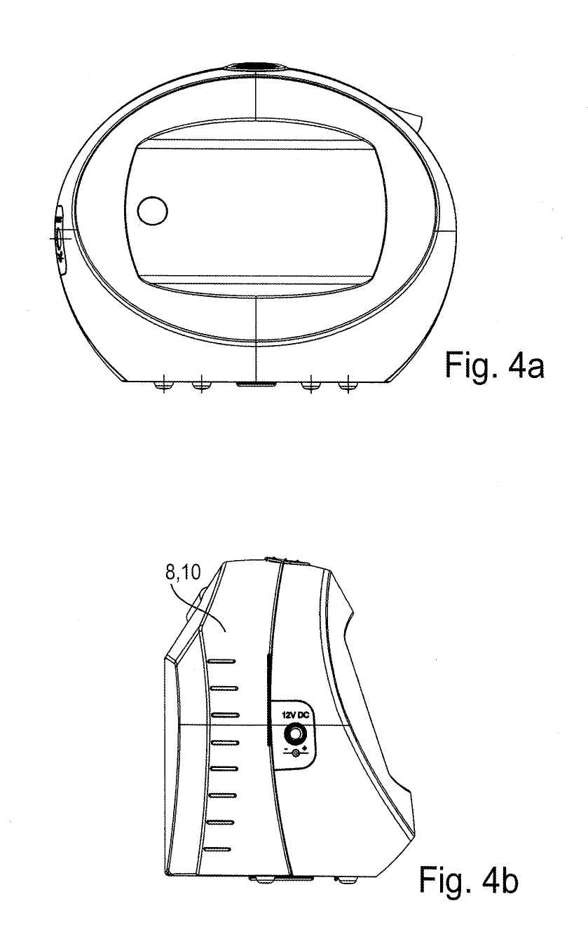

FIGS. 4a through e show views in correspondence with FIGS. 1a through e of a further embodiment of a device, wherein the second housing part is larger than that of the device according to FIGS. 1 through e; and

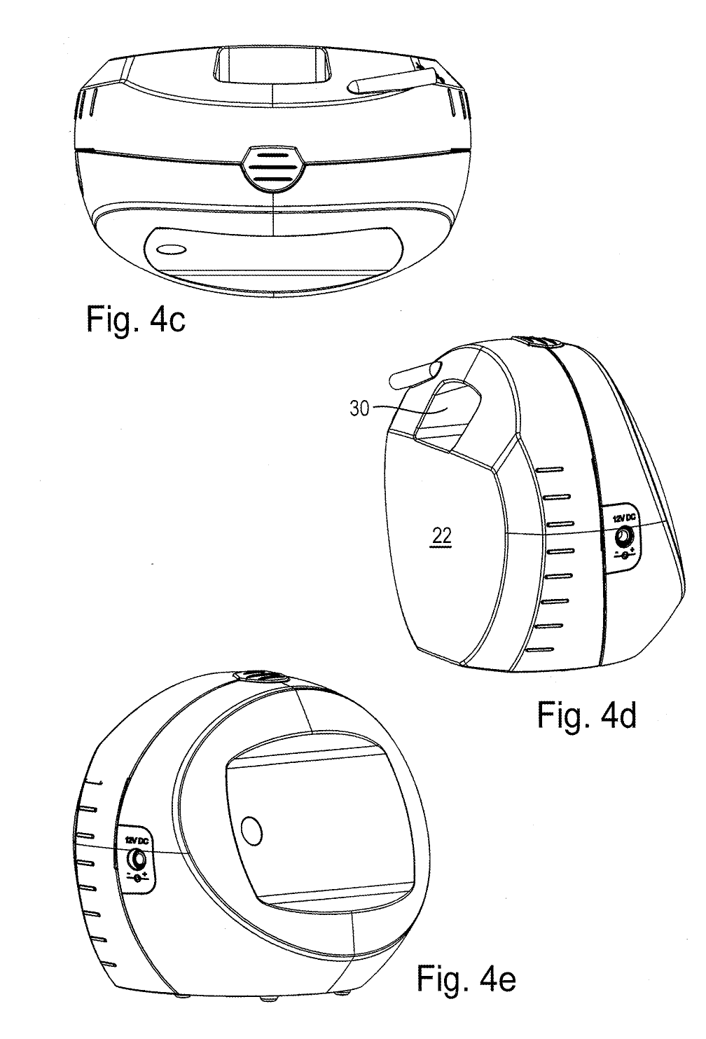

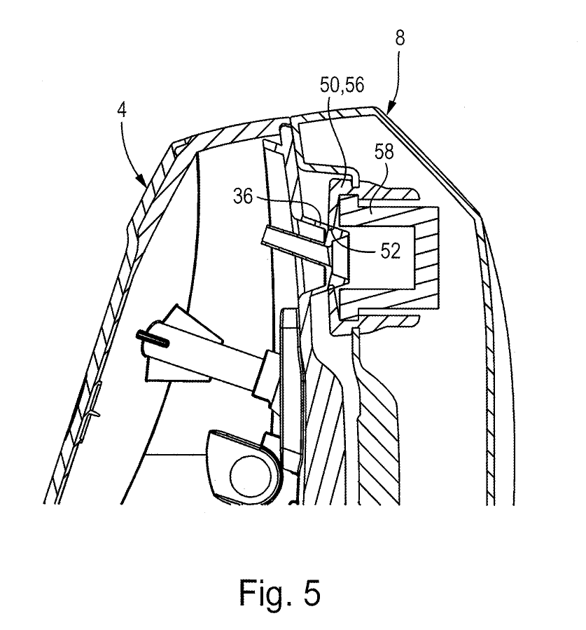

FIG. 5 shows a sectional view through the device in the area of the vacuum communication between the first and the second housing part;

FIG. 6 shows a schematic view of the first and the second housing part with indicated control components.

Two embodiments of a portable device 2 for providing a vacuum for medical applications are initially described with reference to FIGS. 1 through 5, which differ only with respect to size and design of a container for receiving body fluids, which will be described below. The inventive design of the control components of the portable device 2 is subsequently described with reference to FIG. 6.

FIGS. 1a through e show a first embodiment of a portable device 2 for providing a vacuum for medical applications. The device comprises a first housing part 4 which contains a vacuum-generating device in the form of an air suction pump 90 illustrated in FIG. 6 and electrical and electronic control components for the overall device including batteries or advantageously, rechargeable batteries. A charging connection for the batteries is designated by reference numeral 6. The device 2 moreover has a second housing part 8 which forms a container 10 for receiving body fluids, in particular, for receiving exudates extracted from a wound through suction. The overall second housing part 8 is preferably designed as a disposable single-use product. Its upper area is provided with a connecting piece 12 for a suction line 82 shown in FIG. 6, which extends to a wound dressing 80 that seals the wound in a pressure-tight fashion e.g. when the device 2 is used for vacuum therapy of wounds, where the suction line 82 e.g. communicates with the wound area via a port in order to apply and maintain a vacuum in the wound area and extract wound exudate into the container 10 by suction. Towards this end, the container 10 communicates with the suction pump. A connection 13 for an optional measuring or rinsing channel, which extends, like the suction line, to the wound, is also illustrated. This connection extends through the second housing part 8 and terminates in the first housing part 4 from where the measuring and rinsing channel can be loaded e.g. with air as rinsing medium and/or a pressure can be detected and evaluated in this measuring and rinsing channel.

In the illustrated preferred case, the housing parts 4 and 8 rest against each other via a substantially vertical separating plane 14 which is indicated in various figures. When the device 2 is disposed on a flat horizontal support 16, as indicated in FIG. 1a, the separating plane 14 is substantially vertically aligned. This means that the two housing parts 4, 8 are not inserted into one another or stacked on top of each other but come to rest next to each other in the properly assembled state of the device 2. The term separating plane 14 does therefore not mean that it must be a geometrically flat surface, which is directly evident from FIGS. 2a through e, which show different views of the first housing part 4. One can immediately recognize that the side 18 facing the second housing part 8 of the first housing part 4 is not flat at all but has a plurality of elements that project towards the second housing part 8. The side 20 of the second housing part 8 facing the first housing part 4 is substantially complementary to the design of the side 18 of the first housing part 4 such that the two housing parts 4, 8 can only be joined or mounted to each other in a correct fashion. The two housing parts 4, 8 are altogether designed in the form of a disk, i.e. their width B in the horizontal direction and their height H in the vertical direction are each larger than their depth T in the horizontal direction and perpendicular to the width. For this reason, it is possible for the overall device 2 to be designed and sized with respect to its depth such that it can be conveniently carried on the body of a user. In accordance with the invention, the device 2 is designed in such a fashion that the container parts 4, 8 disposed next to each other can be positioned on the body in such a fashion that the second container part 8 faces the body, i.e. comes to rest between the body and the first housing part 4 and the first housing part 4 comes to rest facing away from the body, i.e. substantially forms the visible side of the device 2. For this reason, the side 22 of the second housing part 8 facing the body of the user has a round shape. As can be seen from FIGS. 1c, 1d, 3f, 3e, the side 22 facing the body has a concave shape as viewed in section along a horizontal plane and has, in sections, a radius of curvature R of e.g. 368 mm (FIG. 1c, 3f) in the case illustrated by way of example. The side 22 facing the body additionally also has a concave shape as viewed in section along a vertical plane with a radius of curvature R of e.g. 750 mm (FIG. 1d). In this fashion, the device 2 can be ergonomically arranged and worn in the hip area of a user.

One can also see that an upper area and also a lateral area of the side 22 of the second housing part 8 facing the body are chamfered 24 away from the body of the user towards the first housing part 4 or towards side walls 26 or a peripheral front face of the disk shape of the second housing part 8. In this exemplary case, the chamfer 24 is circumferential. It extends, starting from the base 28, from the bottom to the top, where it extends like an arc to the other side and then back down to the base 28.

FIGS. 1d and 3 also show that an engagement depression 30 in the form of an opening extending through the second housing part 8 is formed on the side 22 of the second housing part 8 facing the body, namely in an upper area of the second housing part 8 that is slightly inclined away from the body. For this reason, the overall device 2 or only its second housing part 8 may be grasped and held by one hand.

In the illustrated preferred embodiment, a manually operable actuator 32, e.g. in the form of a push-button, is provided close to this engagement depression 30 in an upper side of the device 2, which acts on a locking or engaging-behind means 34 (see FIGS. 2b and 2d). In the joined state of the two housing parts 4 and 8, the locking or engagement-behind means 34 is in a locked state, in which the two housing parts 4, 8 are positively held together. The locking is released only by operating the actuator 32 such that the housing parts 4, 8 can be separated from each other. The engagement depression 30 and the manually operable actuator 32 are arranged and designed closely to one another such that a user can engage in the engagement depression 30 and at the same time also operate the actuator 32 with a finger of the same hand, thereby realizing operation with one hand for releasing the second housing part 8 from the first housing part 4. This turns out to be particularly advantageous, since in this case, a second housing part 8 filled with body fluids can be released and discharged into a disposal container using only one hand.

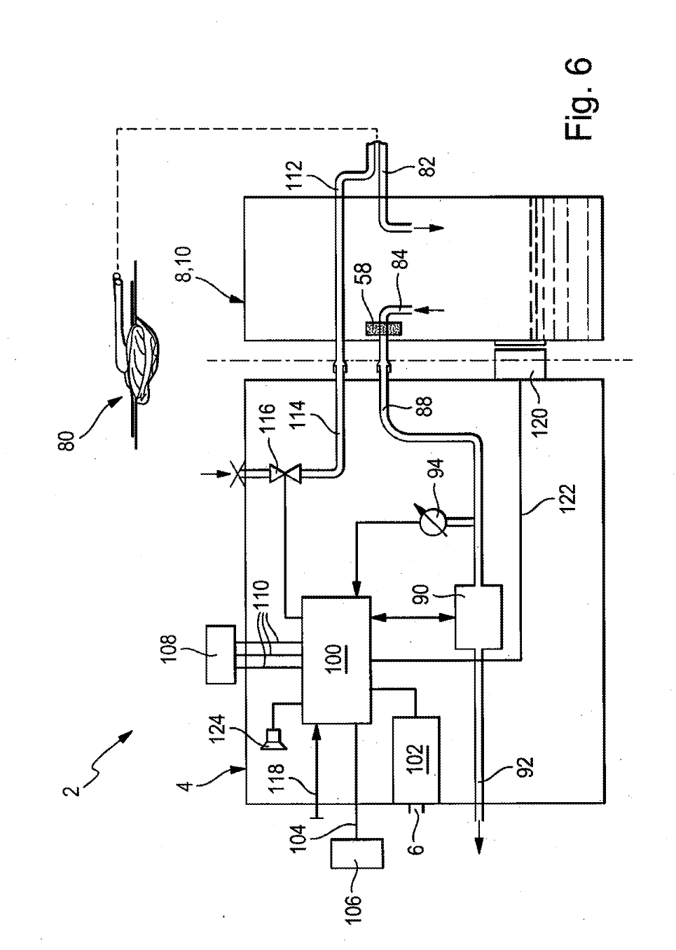

For joining the two housing parts 4, 8, the lower edge of the second housing part 8 is disposed slightly diagonally from the top and from behind onto two pins 33 (FIG. 2d) of the first housing part 4, which form a center of rotation. For this purpose, the second housing part has a recessed area 35 at its lower edge (FIG. 3a) for receiving the pin 33. When the pins 33 and the recessed area 35 are engaged with each other, the second housing part 8 can be pivoted with respect to the first housing part 4. The two facing sides 18, 20 are thereby disposed against each other and thereby move in a self-centering fashion into their intended position (supported by further guiding or centering means 37 (FIGS. 2d) and 39 (FIG. 3a) and the complementary design of the facing sides 18, 20 of the housing parts 4, 8). The locking or engaging-behind means 34 is automatically deflected by movement of the two housing parts 4, 8 relative to each other, in particular, substantially transversely to the vertical separating plane 14, and is then locked in its position locking the housing parts 4, 8 with respect to each other. For this purpose, an engagement hook 41 (FIG. 3i) is provided on the second housing part 8, which is engaged below by the locking means or engaging-behind means 34. When the housing parts 4, 8 are moved into their locked position, a vacuum communication between the inside of the container 10 of the second housing part 8 and the vacuum-generating means is also automatically established through connecting means 36 (described below in connection with FIG. 5).

A visible side 38 of the first housing part 4 facing away from the body is slightly inclined to the vertical such that the disk shape tapers towards the top. In this fashion, the visible side 38 can be better viewed. It shows operating elements 40 and display elements 42, in particular, in the form of a touch screen with a switching foil. The overall visible side 38 is substantially covered or formed by a planar cover 44 such that no gaps are formed in the area of the operating elements 40, which could be soiled.

In the area of the separating plane 14 between the housing parts 4, 8 that rest against each other, the figures moreover show an insertion slot 46 for inserting and detachably fixing a fastening means, in particular and advantageously in the form of a flexible belt, a bracket, a shackle or in any other form, to which e.g. a belt or a shoulder strap can be mounted. It has turned out to be advantageous for the fastening means to be detachable from the housing parts 4, 8 so that it does not disturb when the device 2 is operated in stationary operation, i.e. standing on an advantageously flat support 16, e.g. when a patient to be treated therewith is resting in a hospital bed. In FIG. 2d, means 48 are indicated on the side 18 of the first housing part 4, to/on which the fastening means that are inserted into the insertion slit 46 can be fixed or retained.

The further embodiment of the inventive device illustrated in FIGS. 4a through e differs from the embodiment illustrated in FIG. 3 in that the second housing part 8 and the container 10 formed thereby have a larger volume. The chamfer in the upper area of the side 22 of the second housing part 8 facing the body, where the gripping depression 30 is formed, is inclined a bit further away from the body of the user. This permits even better access. This larger second housing part 8 is rather suited for stationary operation of the device 2. Towards this end, it could also have a side 22 that is convexly curved to the outside or even be more protruding than illustrated in FIG. 4.

FIG. 5 shows in detail the design of the vacuum communication between the inside of the second housing part 8 forming the container 10 and the first housing part 4. The suction side of a vacuum-generating means (not shown) leads to the conically designed connecting means 36 which conically tapers towards the second housing part 8. In this fashion, an at least slightly flexible counter connecting means 50 of the second housing part 8 may be sealingly applied against the conical connecting means 36 of the first housing part 4, which, in the case illustrated by way of example, has a circular opening 62 which is delimited by a resilient sealing lip 54. This counter connecting means 50 terminates in the inside of the second housing part 8. It simultaneously constitutes a filter receiving means 56 for a filter 58 which, in the illustrated exemplary case, is designed as a pot-shaped filter and prevents bacteria from being drawn into the first housing part 4. One can easily recognize that, when the two housing parts 4, 8 are moved relative to each other, the connecting means 36 of the first housing part 4 forms, with the counter connecting means 50 of the second housing part 8, a pressure communication that is sealed to the outside.

In a similar fashion, the coupling between the connection 13 for a measuring or rinsing channel and the associated connecting means 60 that, by way of example, is also designed in a conical shape, is formed on the first housing part 4. As is illustrated in FIG. 3g, a coupling or spout part (not shown) may be inserted into the opening 62 in the second housing part 8 to form the connection 13 for the measuring or rinsing channel illustrated in FIG. 1d. The coupling or spout part (not shown) may then be coupled to the conical connecting means 60 in a pressure-tight fashion. In this fashion, a fluid medium, in particular air or a rinsing liquid, can be guided via a line to the wound for supporting suction of wound exudates. A measuring or rinsing line and the suction line are typically disposable single-use components and are accessories of the second housing part. They are disposed of together with the latter after use.

FIG. 6 shows a purely schematic view of the above-described or a similar device 2 for providing a vacuum for medical applications, wherein corresponding reference numerals are used for corresponding components. FIG. 6, however, only shows the components that are relevant for the following description of the function. FIG. 6 schematically merely indicates a wound to be treated with vacuum and a wound support 80 which is vacuum-tight and to which the suction line 82 from the container 10 is guided. A further line section 84 is guided from the container 10 through the above-mentioned filter 58 to the outside. When the container 10 or the first housing part 8 is moved into its operating position on the first or basic housing part 4 of the device 2, the line section 84 is coupled to a further line section 88 inside the first housing part, which is guided to the suction side of the suction pump 90. This was described above only by way of example with reference to FIG. 5. During operation of the suction pump 90, a vacuum is therefore applied to the container 10 and to the suction line 82 via the line sections 88, 84, and the air suctioned at that location is discharged to the surroundings via an exhaust line 92, wherein shock absorbing elements (not shown) and, if necessary, further filters may additionally be provided.

A pressure sensor 94 is furthermore provided for measuring the pressure in the line section 88 between the container 10 and the suction pump 90. Its signals are entered into a programmable electronic control device which is designated in total by reference numeral 10 and controls or regulates the overall device 2. The above-mentioned charging connection 6 for batteries, which are accommodated in a compartment 102, and a connection 104 for a schematically indicated power unit 106 are also illustrated. Reference numeral 108 designates a display unit with an advantageously provided capacitive switching foil, via which the overall device can be operated. The electrical connection with the electronic control device 100 is only indicated via electric lines 110. The suction pump 90 is driven by the electronic control device 100 in that, by means of the signals of the pressure sensor 94, pressure or vacuum regulation is performed by control and regulation mechanisms (target/performance control mechanisms) known per se, such that the line section 88 is controlled to the pressure value that corresponds to the just selected program.

An additional rinsing or venting line 112 is also illustrated which, in the exemplary case, extends through the container 10 and also leads to the wound cover 80, like the suction line 82. When the container 10 is mounted in its intended mounting position on the first housing part 4, the rinsing line 112 communicates with a line section 114 which is provided in the first housing part 4 and has an electromagnetically operated valve 116, which can be operated by the electronic control device 100 and, in the open state, connects the line section 114 to atmospheric air such that an air flow can be generated via the rinsing line 112 in the direction of the wound.

The device 2 and its electronic control device 100 also have a data interface 118, advantageously a USB interface by means of which the electronic control device 100 or its operation can be programmed.

FIG. 6 also shows the inventive sensor device 120 which cooperates with the electronic control device 100 via merely indicated operating or signal line means 122. This sensor device 120 may be designed in a plurality of ways. The sensor device 120 can detect removal of the container 10 from its intended mounting position on the first housing part 4 during ongoing pressure control operation. This changes in the widest sense the state of the sensor device 120, which can be determined by the electronic control device 100, whereupon the control device 10 then immediately deactivates the suction pump 90.

At the same time, the sensor device 120 may also be used to detect remounting of the container or mounting of a fresh container both prior to start of pressure regulation operation or after deactivation, i.e. interruption of the pressure regulation operation. This change of state of the sensor device 120 is then detected by the electronic control device, which thereupon can automatically trigger restart of operation of the previously interrupted pressure regulation operation in accordance with program technology, wherein in this case, the same operating parameters of pressure regulation operation are used. It would also be feasible to treat remounting of the container in terms of control technology only as an operation release state such that the user himself must initiate restart of the pressure regulation operation.

As mentioned above, it would also be feasible to use the pressure sensor 94 provided for pressure regulation at the same time as a sensor device for detecting removal of a container (in this case, the illustrated separate sensor device 120 would not have to be provided). The removal of the container is then detected on the basis of an abrupt pressure change towards decreasing vacuum, i.e. towards an increasing absolute pressure, preferably in that a pressure change rate (Δp/Δt) determined by means of the signals from the pressure sensor 94 is detected and compared with a predetermined threshold value. When the pressure change rate towards decreasing vacuum is above the threshold value, this can be attributed to advertent or inadvertent release or removal of the container 10 from the first or the base housing part 4. The suction pump 90 is also deactivated in this case via the electronic control device 100. This is advantageously displayed visually via the display unit 108, if necessary, an acoustic signal may alternatively or additionally be emitted via a loudspeaker 124.

In accordance with the invention, an undifferentiated state due to release of the container and, in particular, increased drive of the suction pump is prevented. Operation is consequently performed with defined states and the user gets used to the fact that she/he has to perform restart of the system in case of advertent or inadvertent release of the container, or that this is automatically performed by properly mounting the container.

It has also turned out to be particularly advantageous when the electronic control device 100 does not deactivate the suction pump in case of a vacuum increase in the line section 88 between the container 10 and the suction pump 90, which suggests e.g. an increasing fill level of the container, but continues the predetermined vacuum regulation operation. This vacuum increase, i.e. the decrease of the absolute pressure, may e.g. be only due to the fact that the liquid-tight filter 58 is temporarily blocked due to inclination of the container 10 caused by movements of the user and the container 10 is not yet filled to a level that would require exchange of containers, or a different line could be blocked or pinched off only temporarily. However, irrespective thereof, it has turned out to be advantageous when deactivation of the pressure regulation operation and of the suction pump 90 due to malfunction is realized only when an abrupt pressure increase, i.e. abruptly decreasing vacuum has been determined in the line section 88 between the container 10 and the suction pump 90 which is detected through determination and comparison of the pressure change rate Δp/Δt towards decreasing vacuum with a predetermined threshold value.

1-18. (canceled)

19. A portable device providing a vacuum for medical vacuum treatment of wounds on a body of a person or animal, the device comprising:
a first housing part;
a suction pump disposed in said first housing part, said suction pump generating a vacuum;
a container for receiving body fluids and wound exudate extracted from a wound by suction, said container structured for detachable mounting to said first housing part and for evacuation by said suction pump in a mounted state thereof;
a suction line leading to the body;
a connection cooperating with said container and said suction line for establishing vacuum communication among said suction pump, said container and said suction line;
a pressure sensor;
a programmable electronic control device communicating with said suction pump and said pressure sensor to drive said suction pump, thereby at least taking into consideration predetermined and/or predeterminable parameters as well as pressure values measured by said pressure sensor; and
a sensor device communicating with said electronic control device, said sensor device facilitating detection, by said electronic control device, of removal of said container from said first housing part during vacuum regulation operation,
wherein said sensor device emits a signal, on a basis of which said electronic control device detects removal of said container and initiates deactivation of said suction pump.

20. The device of claim 19, wherein said sensor device has an actuator that is pretensioned towards said container and is mechanically actuated by said container, wherein said actuator is displaced against a pretensioning force when said container is arranged.

21. The device of claim 20, wherein said actuator mechanically operates a switch.

22. The device of claim 19, wherein said sensor device comprises an electric contact sensor.

23. The device of claim 19, wherein said sensor device is magnetically or electromagnetically activated.

24. The device of claim 19, wherein said sensor device is optoelectronically activated.

25. The device of claim 19, wherein said sensor device is designed to be capacitively activated in that a change of a capacitively influenced variable is measured, said variable changing when said container is removed from said first housing part during vacuum regulation operation.

26. The device of claim 19, wherein said sensor device comprises a pressure sensor disposed between said container and said suction pump and structured for measuring a pressure in a line section, said electronic control device being structured to deactivate said suction pump when, starting from ongoing vacuum regulation operation, a pressure change rate (Δp/Δt) towards decreasing vacuum determined by signals of said pressure sensor exceeds a predetermined threshold value.

27. The device of claim 26, wherein said threshold value is 40 mmHg/s or more.

28. The device of claim 19, further comprising an output or display device structured for generating a signal which visually or acoustically communicates removal of said container.

29. The device of claim 19, further comprising an output or display device structured for wireless transmission of a signal to an external receiver, said signal communicating removal of said container to an external receiver.

30. The device of claim 19, wherein said sensor device and said electronic control device are designed in such a fashion that arrangement of said container in an intended position thereof on said first housing part is determined and, starting from a previous deactivation of said suction pump, a previously adjusted pressure regulation operation is restarted.

31. The device of claim 19, further comprising a venting valve controlled by said electronic control device and a venting line connecting said venting valve to a wound area to vent the wound area with external air.

32. The device of claim 19, further comprising an element, controlled by said electronic control device, for supplying a rinsing liquid or another fluid to a wound area via a rinsing line.

33. The device of claim 32, wherein said electronic control device is designed in such a fashion that, upon deactivation of said suction pump, said electronic control device also deactivates said element for supplying a rinsing liquid or another fluid.

34. The device of claim 19, wherein said electronic control device is structured in such a fashion that it does not deactivate said suction pump upon correct or incorrect detection of a full state of said container, rather continues predetermined vacuum regulation operation.

35. The device of claim 19, wherein said electronic control device is designed in such a fashion that, in case of a vacuum increase or reduction of absolute pressure in a line section between said container and said suction pump, said control device does not deactivate said suction pump, rather continues predetermined vacuum regulation operation.

37. A method for operating the device of claim 19, wherein removal of said container from said first housing part during ongoing vacuum regulation operation is detected using the sensor device and the electronic control device, in which case the electronic control device deactivates the suction pump.

38. The device of claim 35, wherein the vacuum increase or the reduction in absolute pressure is in response to a rising fill level of the container.
